(19) United States
(12) Patent Application Publication
(10) Pub. No.: US 2015/0299412 A1
(43) Pub. Date: Oct. 22, 2015

(54) Thiol Acrylate Nanocomposite Foams

(71) Applicants: Daniel HAYES, (US); John Pojman, Baton Rouge, LA (US)

(72) Inventors: Daniel Hayes, Baton Rouge, LA (US); John Pojman, Baton Rouge, LA (US)

(73) Assignee: Board of Supervisors of Louisiana State University and Agricultural and Mechanical College, Baton Rouge, LA (US)

(21) Appl. No.: 14/439,856

(22) PCT Filed: Oct. 31, 2013
(86) PCT No.: PCT/US2013/067663

§ 371 Date: Apr. 30, 2015

Related U.S. Application Data
(60) Provisional application No. 61/721,607, filed on Nov. 2, 2012.

(51) Int. Cl. C08J 9/12; C08J 9/00; A61L 27/46; A61L 27/58; A61L 27/54; C08G 63/688; A61L 27/56

(52) U.S. Cl. CPC C08J 9/122; C08G 63/688; C08J 9/0066; A61L 27/56; A61L 27/58; A61L 27/54; A61L 27/46; C08J 2381/00; A61L 2430/02; A61L 2300/112

(57) ABSTRACT

Thiol-acrylate copolymers are disclosed that are useful, for example, as injectable biomaterials to provide both mechanical support and biological cues to stimulate bone regrowth. Composites can be formed, for example incorporating hydroxyapatite crystal inclusions into the copolymer. In one embodiment, the composite is gas-foamed with a blowing agent during or before cure to form a porous, interconnected scaffold. The synthesis employs an amine-catalyzed Michael addition co-polymerization of a poly-thiol with a poly-acrylate. The catalyst is an in situ catalyst, such as a tertiary amine moiety that is covalently bonded to one of the reactants, preferably to the poly-acrylate. The materials can rapidly co-polymerize in vivo or in vitro via catalysis by the “attached” in situ tertiary amine groups.

The benefit of the Nov. 2, 2012 filing date of U.S. provisional patent application Ser. No. 61/721,607 is claimed under 35 U.S.C. §119(e) in the United States, and is claimed under applicable treaties and conventions in all countries. The complete disclosure of the priority application is hereby incorporated by reference in its entirety.

This invention was made with government support under grant number 2012-31100-06022/LA94093 awarded by the United States Department of Agriculture, National Institute of Food and Agriculture (NIFA). The United States Government has certain rights in this invention.

### TECHNICAL FIELD

This invention pertains to thiol-acrylate copolymers, thiol-alkyne copolymers, copolymer/ceramic composites, and their use as biocompatible and absorbable materials for tissue repair and regeneration, particularly bone repair and regeneration.

### BACKGROUND ART

Bone grafts have been the standard treatment to augment or accelerate bone regeneration for decades. Autogenous cancellous bone grafts have been used to facilitate bone regrowth, although quantity is limited and surgical procedures for graft harvest are required. Allogeneic bone grafts are costly, require time-consuming bone banking procedures, have high potential for disease transmission, and can result in host-versus-graft disease. Neither technique provides a clinically convenient method for conformal filling of a critical sized bone defect, i.e., a defect whose size is such that the defect will not readily heal spontaneously.

Engineered synthetic bone tissue has emerged as an alternative to allogeneic or autogenic bone grafts. This field will become an important alternative to current surgical techniques to replace or restore the function of traumatized, damaged, or lost bone. Desirable features of an engineered composite bone scaffold include a biodegradable composition, broad biocompatibility, large pore volume, and osteoinductive/conductive properties.

Native bone contains hydroxyapatite crystals (HA) distributed within an organic matrix. The porosity and degree of mineralization vary among different bone types.

Many biodegradable materials have been explored as possible bone scaffold substrates, including various natural products, gelatin/bioactive ceramics, and poly(α-hydroxyesters). Several studies have reported the use of extracellular matrix or other naturally occurring compounds to engineer bone tissue, such as collagen, decellularized bone, or chitosan. Gelatin/bioactive ceramics, such as calcium and magnesium phosphates, have been studied as well.

Synthetic hydroxyapatite composites with degradable polymers such as poly-L-lactic acid, (PLLA) polyglycolic acid (PGA), or poly-ε-caprolactone (PCL), have been used in hybrid cell/scaffold constructs. Scaffold constructs combined with mesenchymal stromal/stem cells (MSC) have resulted in improved bone formation.

The fabrication method itself can have a substantial impact on the mechanical properties and the function of the scaffold, affecting pore size, pore volume, and void interconnectivity. In turn, these factors influence cell attachment, proliferation, extracellular matrix production, and the transport of nutrients and wastes. Common approaches include solid freeform fabrication, thermal precipitation, gas foaming, and solvent casting (often followed by particulate leaching).

Thiol-ene chemistries such as thiol-acrylate chemistries have been used in tissue engineering applications. When a thiol-acrylate chemistry is used, all or nearly all of the materials used in the synthesis are incorporated into the complete, finished network, thus reducing the risks of leaching toxic monomers or short chain oligomers. Additionally, bioactive compounds such as peptides, proteins, enzymes, oligonucleotides, and nucleic acids can be copolymerized with thiol-acrylate chemistries. Previous thiol-acrylate chemistries used in biomedical applications have been photolytically polymerized using radical-based photoinitiators.

X. He et al., “Material Properties and Cytocompatibility of Injectable MMP Degradable Poly(lactide ethylene oxide fumarate) Hydrogel as a Carrier for Marrow Stromal Cells,” Biomacromolecules, vol. 8, pp. 780-792 (2007) discloses an in situ cross-linkable poly(lactide-co-ethylene oxide-co-fumarate) macromer developed with ultralow molecular weight poly(L-lactide) and poly(ethylene glycol) (PEG) units, linked by fumaryl units, and their use as injectable, crosslinkable biomaterials for purposes such as treating bone defects. The macromer was crosslinked with a degradable peptide sequence containing acrylate end-groups or a methylene bisacrylamide crosslinker to form enzymatically or hydrolytically degradable hydrogels.

C. Nuttleman et al., “The effect of ethylene glycol methacrylate phosphate in PEG hydrogels on mineralization and viability of encapsulated hMSCs,” Biomaterials, vol. 27, pp. 1377-1386 (2006) discloses the incorporation of photoreactive, phosphate-containing ethylene glycol methacrylate phosphate into poly(ethylene glycol) hydrogels, to improve the adhesion of human mesenchymal stem cells in the hydrogel.

See also Schneider G B, English A, Abraham M, Zaharias R, Stanford C, Keller J. “The effect of hydrogel charge density on cell attachment,” Biomaterials 2004; 25(15):3023-3028; Kumar D, Gittings J P, Turner I G, Bowen C R, Bastida-Hidalgo A, Cartmell S H. “Polarization of hydroxyapatite: Influence on osteoblast cell proliferation,” Acta Biomaterialia 2010; 6(4): 1549-1554; Salinas et al., “Mixed Mode Thiol-Acrylate Photopolymerizations for the Synthesis of PEG-Peptide Hydrogels,” Macromolecules 2008, 41, 6019-6026; and Rydholm, A. E.; Reddy, S. K.; Anseth, K. S.; Bowman, C. N., “Controlling network structure in degradable thiol-acrylate biomaterials to tune mass loss behavior,” Biomacromolecules 2006, 7, 2827-2836.

There is an unfilled need for improved compositions and methods for bone tissue engineering.

### DISCLOSURE OF THE INVENTION

We have discovered novel thiol-acrylate copolymers that are useful, for example, as injectable biomaterials to provide both mechanical support and biological cues to stimulate bone regrowth. We have discovered a novel method for synthesizing the novel copolymers, and novel composites that incorporate hydroxyapatite crystal (HA) inclusions into the copolymer. In a preferred embodiment, the novel composite is gas-foamed with a blowing agent during or before cure to form a porous, interconnected scaffold. The novel synthesis employs an amine-catalyzed Michael addition co-polymerization of a poly-thiol with a poly-acrylate. The catalyst is an in situ catalyst, such as a tertiary amine moiety that is covalently bonded to one of the reactants, preferably to the poly-acrylate. The materials can rapidly co-polymerize in vivo or in vitro via catalysis by the “attached” in situ tertiary amine groups.

One embodiment of the novel synthesis is depicted in FIGS. 1A through 1D. Although particular reagents are depicted in FIGS. 1A-1D for purposes of illustration, more generally the reagents in the novel synthesis are: (1) a poly-acrylate containing two or more acrylate groups, preferably a tri-acrylate; (2) a poly-thiol containing two or more thiol groups, preferably a tri-thiol; and (3) ammonia, a primary amine, a secondary amine, or a tertiary amine, preferably a secondary amine.

At least tri-functionality in the thiol or the acrylate is needed for crosslinking. Preferably, both the thiol and the acrylate are trifunctional. Using ammonia or a primary amine rather than a secondary amine in the initial step of forming the in situ catalyst also enhances crosslinking, as it effectively increases the number of acrylate groups in those molecules that contain an amine center after the in situ tertiary amine catalyst is formed.

The steps of one embodiment of the in situ amine-catalyzed, anionic step growth polymerization mechanism are illustrated in FIGS. 1A through 1D. The first step, depicted in FIG. 1A, is the formation of an in situ catalyst/comonomer molecule through the Michael addition of a secondary amine across the carbon-carbon double bond of one of the acrylate groups in the tri-acrylate monomer. The rate of the later polymerization steps can be tuned by adjusting the percentage of acrylate groups that react with an amine functionality. In prototype embodiments to date, this percentage has typically been on the order of ˜1%, but the percentage may be adjusted up or down to fit particular needs. This percentage is preferably between about 0.1% and about 10%. The amine starting material as depicted in FIG. 1A is a secondary amine. By instead employing a primary amine or even ammonia, the resulting secondary or primary amine product can continue to react with other thiol groups until a tertiary amine-thiol product results, thereby increasing the level of cross-linking (if enhanced cross-linking is desired).

The second step, depicted in FIG. 1B is to react the “activated” acrylates (i.e., those incorporating an in situ amine catalytic group) with a thiol comonomer (e.g., TMPTMP as depicted in FIG. 1B). The amine abstracts a hydrogen from a thiol group, producing a thiol anion.

As depicted in FIG. 1C, the reaction then proceeds via an anionic chain reaction mechanism with a sequential chain transfer step after each addition. An alternative propagation route is depicted in FIG. 1D.

Because the in situ catalyst is a tertiary amine, the novel synthesis has no possibility for leaching potentially toxic free radical initiators, as would be the case with a more typical radical polymerization mechanism. Unreacted monomer and unreacted amine may remain in low concentrations embedded within the polymer product, but they would not be expected to be especially reactive once chain propagation has ended; and furthermore their molecular weights are typically such that they would tend to diffuse from within the polymer product only slowly, and therefore they should not present a substantial risk in vivo. Thus, this chemistry is more attractive for in situ polymerization in a bone defect than are comparable free radical-based methods. The novel copolymer products themselves differ from copolymers that could be synthesized by free radical reaction, in that there is more control over the nature and degree of cross-linking. For example, using free-radical polymerization, it is not possible to create a true 1:1 copolymer of a thiol and an acrylate, as is possible with the novel technique. Moreover, free-radical polymerization produces a crosslinked network that is generally less uniform than the networks that can be produced through step-grown polymerization of the thiol-acrylate. Another advantage of the novel system is that little or no initiator decomposition products remain that might diffuse from the product into surrounding biological tissue.

Optionally, hydroxyapatite can be added to the copolymerization mixture before or during curing to impart mechanical strength, and to provide osteogenic materials for promoting new bone growth. Alternatively, a long-chain monomer such as a polyethylene glycol triacrylate or other multifunctional acrylate may be used to produce a product that is useful for soft tissue repair.

Optionally, other naturally-occurring or synthetic molecules may be incorporated into the copolymer during the polymerization step, preferably by incorporating thiol-containing molecules into the polymerization mixture. For example, an antibody, peptide, enzyme, or protein with at least one free thiol group (e.g., from a cysteine residue) can react with the growing polymer at some locations in lieu of the principal polymerization reaction. Or a cysteine residue may be added to either end of a peptide or protein that may not otherwise have a suitable cysteine available for such a reaction. Similarly, a thiol-, alkene-, or alkyne-terminated oligonucleotide or nucleic acid sequence could be incorporated into the polymer during polymerization via the Michael addition mechanism. These options add considerable flexibility to the uses and properties of the copolymer product.

We have synthesized and characterized several members of the novel class of thiol-acrylate copolymers. Their mechanical properties, cytocompatibility, and osteogenic properties are desirable for stimulating bone regrowth. Their gelation times are tunable, providing flexibility for material preparation and delivery. A series of biocompatibility experiments have demonstrated that the new synthetic polymers are capable of supporting human adipose-derived stromal cell (hASC) cultures, which were chosen as a model cell type for demonstrating cytocompatiblility and osteogenic behavior. Metabolic activity, calcium deposition, DNA content, and the expression of the osteogenic markers alkaline phosphatase and osteocalcin in hASC were measured for different scaffold compositions.

SEM and micro-CT images have shown the morphology of solid and foamed pentaerythritol triacrylate mixed with thiol comonomer (PETA-co-TMPTMP) composites, with varying HA concentrations. SEM imaging of hASC showed a spindle-shaped morphology, indicating adherence of the cells to the copolymer. Micro-CT analysis showed open-cell, interconnected pores.

### BRIEF DESCRIPTION OF THE DRAWINGS

FIGS. 1A-D depict the mechanism for a representative in situ, tertiary amine-catalyzed, anionic step growth polymerization in accordance with the present invention.

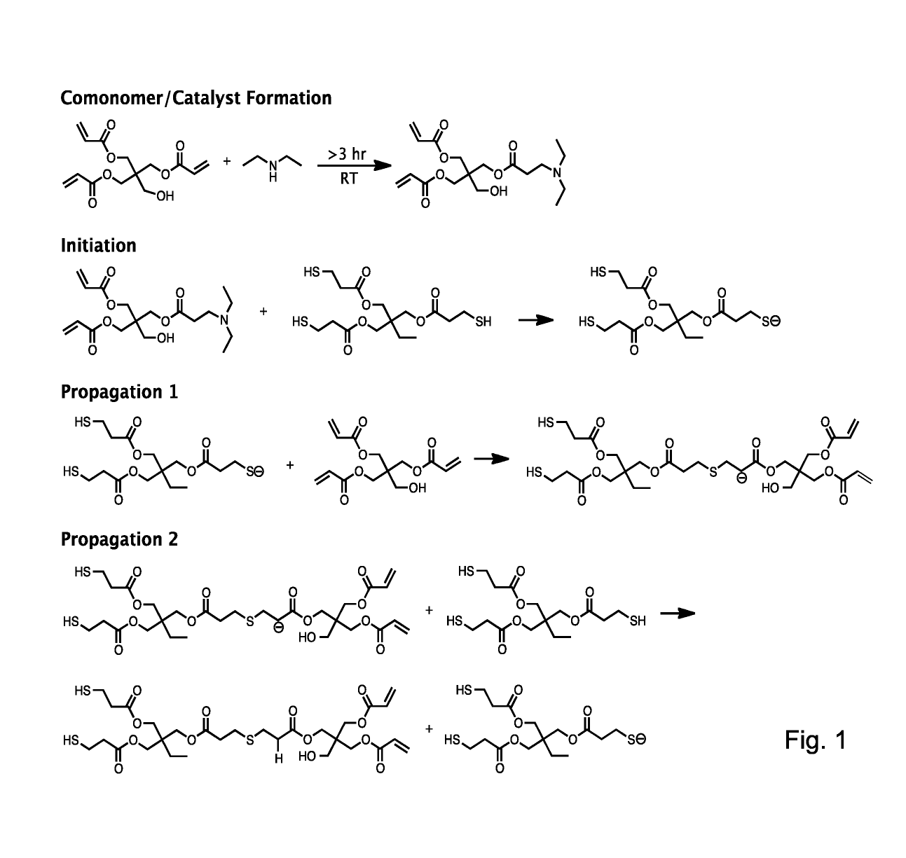

FIG. 2 depicts mass loss of various polymers after 7 days of incubation in stromal control media.

FIG. 3 depicts mass loss of various solid cast samples and foamed samples after 7 days of incubation in stromal control media. For the HA composite samples, the HA content was 20%.

FIG. 4 depicts the relative metabolic activity of hASC in thiol-acrylate extracts as measured by AlamarBlue™ fluorescent conversion. For the HA composite samples, the HA content was 20%.

FIG. 5 depicts relative metabolic activity of hASC exposed to 7 days of stromal control media extracts. For the HA composite samples, the HA content was 20%.

FIG. 6 depicts the compressive yield strength measured in mechanical tests of foamed and solid composites.

FIG. 7 depicts relative metabolic activity for hASC cultured on different solid composites. For the HA composite samples, the HA content was 20%.

FIG. 8 depicts relative metabolic activity for hASC cultured on foamed composites. For the HA composite samples, the HA content was 20%.

FIG. 9 depicts metabolic activity for hASC cultured on various scaffolds.

FIG. 10 depicts calcium deposition for cells cultured on various scaffolds in stromal and osteogenic media.

FIG. 11 depicts a quantitative measure of hASC proliferation on various scaffolds, using PicoGreen™ to determine total DNA content.

FIG. 12 depicts differences in the expression of alkaline phosphatase (ALP) and osteocalcin (OCN) in hASC cultured on various scaffolds in stromal and osteogenic media. The vertical axis depicts the observed fold-change.

FIGS. 13A-D depict SEM images of in vitro PETA-co-TMPMP foam and in situ PETA-co-TMPTMP foam.

FIGS. 14A-C depict micro-CT images of orthogonal slices of foamed scaffold samples.

### MODES FOR CARRYING OUT THE INVENTION

The compositions of this invention have broadly tunable mechanical and chemical properties. Various biocompatible polymer and copolymer compositions containing thiol, and acrylate or alkyne moieties within the scope of the invention can be synthesized using the same general method and reaction scheme. By varying the number of functional moieties one may synthesize straight chain, branched, or cross-linked compositions. For practical in situ polymerization, it is preferred to have gel times that are tunable across a wide range of times (typically, from minutes to hours). Such tunability is readily achieved with the thiol-acrylate system of this invention. For example, the rate of reaction may be tuned by adjusting the concentration of in situ amine functionality. The properties of the product may be tuned, for example, by modifying the degree of crosslinking, or by incorporating substituents into the thiol or acrylate comonomer. Although an acrylate is the preferred co-monomer, alkynes may be used as the co-monomer as well. Depending on the degree of cross-linking desired, the number of alkyne groups in the molecule may be varied from one to two to three or even more. As another alternative, a mixture of acrylate and alkyne may be employed.

### MATERIALS AND METHODS

### Example 1

### Materials

Unless otherwise stated, the stromal control media used in the prototype demonstrations comprised Dulbecco's Modified Eagle's Medium, 10% fetal bovine serum, and 1% triple antibiotic solution. Unless otherwise stated, the osteogenic media used in the prototype demonstrations comprised Dulbecco's Modified Eagle Medium, 10% fetal bovine serum, 1% triple antibiotic solution, 0.1 μM dexamethasone, 50 μM ascorbate-2-phosphate, and 10 mM β-glycerophosphate.

All reagents were used as received.

| Material | Abbreviation | Note | Supplier |
|---|---|---|---|
| poly(ethylene glycol) diacrylate | PEGDA | MW 700 and | Aldrich |
| | | MW 575 | |
| trimethylolpropane ethoxylate triacrylate | TMPETA | MW 912 and | Aldrich |
| | | MW 692 | |
| trimethylolpropane triacrylate | TMPTA | | Aldrich |
| poly-ε-caprolactone | PCL | | Aldrich |
| trimethylolpropane tris(3-mercaptopropianate) | TMPTMP | | Aldrich |
| pentaerythritol triacrylate | PETA | | Alfa Aesar |
| diethylamine | DEA | 99% purity | AGROS Organics |
| hydroxyapatite crystals | HA | | |
| human adipose-derived stromal cells | hASC | | |
| Stromal control media: Dulbecco's Modified | | | |
| Eagle's Medium, 10% fetal bovine serum, 1% | | | |
| triple antibiotic solution | | | |
| Osteogenic media: Dulbecco's Modified | | | |
| Eagle's Medium, 10% fetal bovine serum, 1% | | | |
| triple antibiotic solution, 0.1 μM | | | |
| dexamethasone, 50 μM ascorbate-2-phosphate, | | | |
| 10 mM β-glycerophosphate | | | |
| AlamarBlue ™ | | | Invitrogen ™ |
| PicoGreen ® dye solution | | 0.1 g/mL | Invitrogen ™ |
| alizarin red stain | | | |
| phosphate buffered saline | | | |
| Hank's balanced salt solution | | | |
| cDNA EcoDry Premix | | | ClonTech |
| 2X iTaq ™ SYBR ® green supermix with ROX | | | Bio-Rad |
| 2% glutaraldhyde: 2 parts Cocadylate, 1 part | | | |
| 8% glutaraldhyde, 1 part distilled H₂O | | | |
| hexamethyldisilazane | | | |
| 1,4-dioxane | | | |
| carbon dioxide | CO₂ | | |
| compressed nitrous oxide | | | |
| 0.9% sodium chloride | NaCl | | |
| 70% ethanol | | | |
| 10% cetylpyridinium chloride monohydrate | | | |
| proteinase K | | | |

### Example 2

### Preparation of Thiol-Acrylate Compositions

Several compositions containing TMPTMP with di- or tri-functional acrylates (listed in Example 1) were prepared in a 1:1 functionality ratio. These compositions were subjected to mass loss and hASC cytotoxicity tests (explained in Examples 4 and 5). Twenty stock solutions containing PETA, a preferred acrylate in terms of biocompatibility and mass loss data, and DEA (content ranging from 2.8-35.1%) were prepared, and the resulting copolymer compositions were subjected to mechanical testing (Example 6).

The strength of the materials can be altered by varying the initial DEA concentration. The functionality and thus the cross-linking density are functions of the amine concentration. The first Michael addition reaction with the secondary amine results in a loss of one acrylate functionality from the trifunctional acrylate.

A 16.1% DEA concentration was chosen for use in a preferred bone repair composition because it produced the highest Young's Modulus as measured in our initial experiments. The composition had high elasticity and high mechanical strength. A preferred gel time for many applications is on the order of 15-20 minutes, to allow time to mix and apply the material, while forming a material with suitable flexural strength within a practical time. The gel time can be tuned by adjusting the concentration of amine, and the number of functional groups on the thiol or acrylate comonomer.

### Example 3

### Preparation of Foamed and Solid Materials

DEA and PETA were used to prepare a solution for both foamed and solid composite materials. The DEA and PETA (16.1% DEA by acrylate molar functionality) were combined in advance, and stirred for 24 hours to form the in situ catalyst/comonomer. TMPTMP was added in a 1:1 molar functionality ratio (i.e., the ratio of thiol groups to acrylate groups), and the material was mixed with a stir rod for 3 hours. Then several concentrations of HA (0%, 15%, 20%, 25% wt/wt) were added to the PETA-co-TMPTMP solution. This solution was cast into cylindrical molds (10×10 mm), and allowed to cure for 24 hours to form the solid composite copolymer material.

A foamed composite copolymer was prepared by pouring the PETA-co-TMPTMP with HA (0%, 15%, 20%, 25% wt/Wt) into a 250 mL pressurized spray canister using 7 g compressed nitrous oxide as a gas foaming agent. The mixture was expelled from the canister into the same types of cylindrical, polydimethylsiloxane molds as those used for the solid casting.

A PETA-co-TMPTMP+20% HA foamed sample was prepared in vitro by foaming directly into a beaker containing stromal control media (instead of the cylindrical molds) to observe the effect of physiological conditions on polymerization and foam structure. There was no inhibition of polymerization from the stromal aqueous media.

PCL+0% HA and PCL+20% HA foams were fabricated by thermally-induced phase separation from 1,4-dioxane, followed by lyophilization, as otherwise described in Zanetti A S, McCandless G T, Chan J Y, Gimble J M, Hayes DJ. Characterization of novel akermanite:poly-ε-caprolactone scaffolds for human adipose-derived stem cells bone tissue engineering. Journal of Tissue Engineering and Regenerative Medicine Accepted Oct. 4, 2012.

The experimental groups were the following:

PETA-co-TMPTMP+0% HA solid copolymer

PETA-co-TMPTMP+0% HA foam copolymer

PETA-co-TMPTMP+15% HA solid copolymer

PETA-co-TMPTMP+15% HA foam copolymer

PETA-co-TMPTMP+20% HA solid copolymer

PETA-co-TMPTMP+20% HA foam copolymer

PETA-co-TMPTMP+25% HA solid copolymer

PETA-co-TMPTMP+25% HA foam copolymer

PETA-co-TMPTMP+20% HA foam copolymer in vitro

PCL+0% HA solid polymer

PCL+0% HA foam polymer

PCL+20% HA solid polymer

PCL+20% HA foam polymer

### Example 4

### Mass Loss Testing

The mass of both foamed and solid composite polymers were measured. The PCL foam polymers served as positive controls, and PETA-co-TMPTMP+0% HA served as an internal comparison. All samples were normalized versus the initial mass before media exposure. The samples were incubated on an orbital shaker with 5 mL stromal control media at 37° C., 200 rpm for 7 days. The samples were tested by the multiple headspace extraction method to determine mass loss. The resulting extracts were used in cytotoxicity testing.

### Example 5

### Cytotoxicity Testing

The extracts from the mass loss test were filtered (0.22 μm pore size) and pipetted (100 μL/well) into a 96-well plate previously sub-cultured with hASC (2,500 cells/well). The plates were placed in an incubator at 37° C. under 5% CO₂ for 24 hours. The cellular viability on the scaffolds was determined by adding 10 μL AlamarBlue reagent to each well, and re-incubating at 37° C. under 5% CO₂ for 2 hours. Fluorescence was measured at an excitation wavelength of 530 nm and an emission wavelength of 595 nm using a fluorescence plate reader. A tissue culture-treated plastic 96-well plate served as a control substrate.

### Example 6

### Mechanical Testing

Compression testing was performed on four specimens of each scaffold type with cylindrical geometry of 10×10 mm at room temperature using a hydraulic universal testing machine (Instron Model 5696, Canton, Mass., USA) at an extension rate of 0.5 mm/min, to a maximum compression strain of 90%. The scaffolds tested were solid and foamed PETA-co-TMPTMP containing HA (0%, 15%, 20%, 25% wt/wt), and in vitro foamed PETA-co-TMPTMP+20% HA. For comparison, the measured compressive strengths of human cortical and cancellous bone are 130-180 MPa and 4-12 MPa, respectively.

### Example 7

### Isolation and Culture of hASC

Liposuction aspirates from subcutaneous adipose tissue were obtained from three healthy adult subjects (1 male, 2 female) undergoing elective procedures. All tissues were obtained with informed consent under a clinical protocol reviewed and approved by the Institutional Review Board at the Pennington Biomedical Research Center. Isolation of hASC was performed as otherwise described in Gimble J M, Guilak F, Bunnell BA. Clinical and preclinical translation of cell-based therapies using adipose tissue-derived cells. Stem Cell Research & Therapy 2010; 1.

### Example 8

### hASC Loading on Scaffolds and Culture

In Passages 0 and 1 of the primary cell culture, cells were passaged after trypsinization and plated at a density of 5,000 cells/cm², and were then expanded on T125 flasks to 80% confluence.

In one embodiment, Passage 2 of an individual cell culture was used for a cell viability test after acute exposure to the scaffold media extracts on loaded scaffolds in a spinner flask.

In another embodiment, Passage 2 of an individual cell culture was used to evaluate in vitro hASC osteogenesis on tissue culture-treated polymers for different scaffold types.

In both embodiments, 5.0×10⁴ cells (Passage 2)/5 μL from each donor were pooled and loaded on the top of each sample. After 30 minutes of incubation at 37° C. and 5% CO₂, the opposite side of each sample was similarly loaded with the same number of cells. Loaded scaffolds were subsequently transferred to 48-well plates and cultured in stromal control media or in osteogenic media for 21 days. Media maintenance was performed three times a week, and samples were collected weekly.

### Example 9

### In Vitro hASC Viability and Metabolic Activity on Scaffolds

The ability of the PETA-co-TMPTMP copolymer to support hASC adhesion and short-term culture was evaluated using AlamarBlue metabolic activity assays and examining cell morphology. (AlamarBlue is an indicator of cell viability and proliferation.)

The viability of cells within the scaffolds in stromal control media was determined after 7 days using an AlamarBlue metabolic activity assay. The metabolic activity of cells within the scaffolds in stromal or osteogenic media was also determined using the AlamarBlue assay at 7, 14, and 21 days. To perform these assays, the scaffolds were removed from culture, washed three times in phosphate buffered saline, and incubated with 10% AlamarBlue in Hank's balanced salt solution without phenol red (pH 7) for 90 minutes. Aliquots (100 μL) of AlamarBlue in Hank's balanced salt solution were placed in a 96-well plate in triplicate, and fluorescence was measured at an excitation wavelength of 530 nm and an emission wavelength of 595 nm using a fluorescence plate reader.

### Example 10

### In Vitro Calcium Deposition

Alizarin red staining, which stains calcium-rich deposits, was used to assess the osteogenic potential of different scaffold types. hASC osteogenesis (either for cells alone, or for cells with scaffolds) was assessed after 7, 14, and 21 days of culture in stromal or osteogenic media using alizarin red stain. Cells and scaffolds were briefly washed four times with 0.9% NaCl (1 mL/well) and fixed with 70% ethanol (1 mL/well). The fixative was removed by aspiration; plates were stained with 2% alizarin red for 10 minutes, and then washed with deionized water six times. To remove stain from the samples, 400 mL of 10% cetylpyridinium chloride monohydrate was added to each well, and the samples were shaken for 10 minutes at room temperature. Calcium deposition was then measured by optical density at 540 nm with a plate reader. Scaffolds cultured without cells were used as controls to normalize the results.

### Example 11

### In Vitro Quantification of DNA on Scaffolds

Total DNA content was used to estimate the number of cells on each scaffold, following the protocol otherwise described in Karageorgiou V, Kaplan D. Porosity of 3D biomaterial scaffolds and osteogenesis. Biomaterials 2005; 26(27):5474-91 and Van Blitterswijk C A, Hesseling S C, Grote J J, Koerten H K, de Groot K. The biocompatibility of hydroxyapatite ceramic: a study of retrieved human middle ear implants. J Biomed Mater Res 1990; 24(4):433-53. The scaffolds were crushed, and the DNA was digested with 0.5 mL proteinase K (0.5 mg/mL) at 56° C. overnight. Aliquots (50 μL) from the digested mixtures were mixed with equal volumes of PicoGreen dye solution (0.1 g/mL) in 96-well plates. Fluorescence measurements were then taken with the samples excited at 480 nm. Scaffolds without cells were used as negative controls.

### Example 12

### Quantitative Reverse Transcriptase Polymerase Chain Reaction

Total RNA was extracted using TRIzol™ reagent. Total RNA to cDNA EcoDry Premix was used in the cDNA synthesis. A quantitative polymerase chain reaction was performed using 2× iTaq SYBR green supermix with ROX, and primers specific for alkaline phosphatase and osteocalcin. Osteogenic differentiation was assessed with hASC loaded in scaffolds and cultured in stromal or osteogenic media for 7, 14, and 21 days. Reactions were performed with an MJ Mini™ Thermal Cycler (Bio-Rad). Water (negative) and hASC culture in osteogenic media for 14 days (positive) were used as controls. Samples were normalized (ACt) against measured values for the housekeeping gene 18S rRNA. The fold change expression of alkaline phosphatase and osteocalcin in scaffolds cultured in stromal and osteogenic media was calculated using the ΔΔCt method, as otherwise described in Liu, Q.; Cen, L.; Yin, S.; Chen, L.; Liu, G.; Chang, J.; Cui, L., “A comparative study of proliferation and osteogenic differentiation of adipose-derived stem cells on akermanite and β-TCP ceramics” Biomaterials 2008, 29, 4792-4799.

### Example 13

### SEM Analysis

Each scaffold type was imaged by SEM. Solid PETA-co-TMPTMP copolymer samples were placed in a 12-well plate to form a thin layer (1 mm thick). The polymers that had been seeded with stem cells were fixed for 30 minutes with 2% glutaraldhyde. Then all samples were dehydrated by washing with ethanol, starting with a 30% ethanol solution, increasing by 10% every 30 minutes to 100% ethanol. Overnight 100% hexamethyldisilazane was added to the samples to replace the dried air and ethanol. An EMS550X sputter coater applied a conductive platinum coating for 2 minutes, followed by standard SEM imaging. SEM images were also taken of in situ and in vitro foamed samples.

### Example 14

### Micro-CT Analysis

Four PETA-co-TMPTMP foams were fabricated with pressurized extrusion foams containing different levels of HA (0%, 15%, 20%, 25% wt/wt). Another sample containing PETA-co-TMPTMP+20% HA content was foamed into stromal control media. Samples were sliced into 1-2 mm approximate cuboids of 10-15 mm height. Samples were imaged with 11 or 13 keV monochromatic x-rays with 2.5 μm/px resolution at the tomography beamline in the Center for Advanced Microstructures and Devices (Louisiana State University, Baton Rouge, La.). Projections numbered 720, corresponding to Δθ=0.25. Projection exposure time varied between 2 and 4 seconds, and reconstruction algorithms ensured normalized data. The two different datasets are directly comparable, both as an aggregate dataset and as slices. Reconstruction data are 16-bit signed integer with mean air intensity scaled to zero. Pore size was measured using ImageJ 64.

Volume renderings were generated from three-dimensional data of foamed samples using Avizo Fire software, version 7.0.1 (Visualization Services Group). Two overlapping sub-volumes were rendered simultaneously, one with a red-orange-white color map corresponding to thiol-acrylate foam, and another with a blue-green color map corresponding to hydroxyapatite inclusions. Orthogonal slices were created using ImageJ, with equivalent scale, brightness, contrast, and grey map settings.

### Example 15

### Statistical Analysis

All results were expressed as mean±SEM. Normality of the data was confirmed using the Shapiro-Wilk test (P<0.001). Data were analyzed with one or two-way analysis of variance (ANOVA), followed by Tukey's minimum significant difference post hoc test for pairwise comparisons of main effects. For all comparisons, a P-value<0.05 was considered significant.

Results and Discussion

### Example 16

### Mass Loss Results

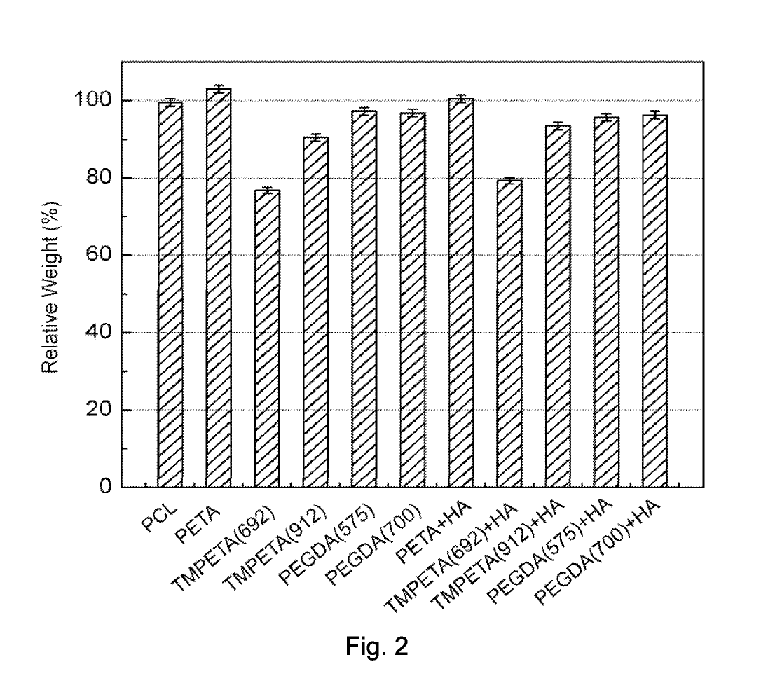

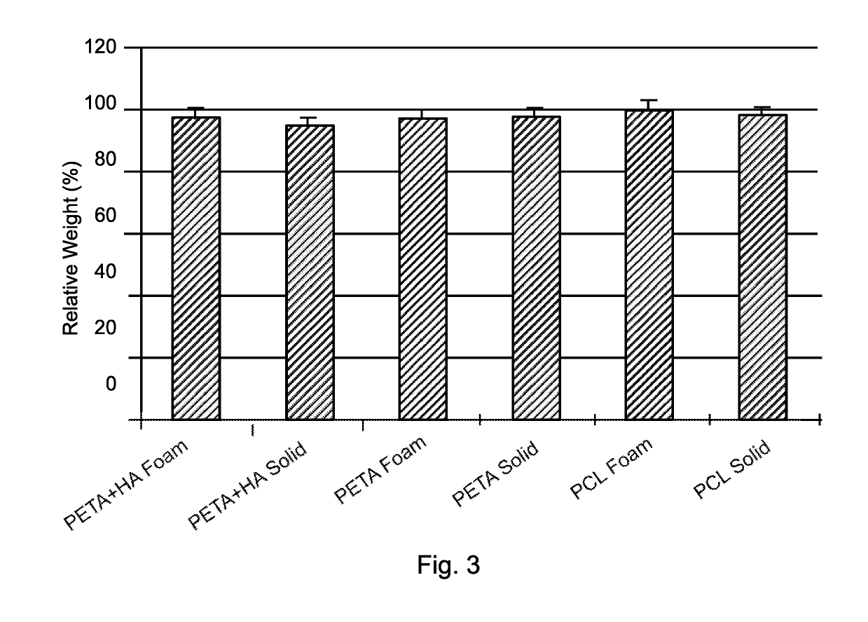

Mass loss after 7 days of incubation in stromal control media is illustrated in FIGS. 2 and 3. As FIG. 2 illustrates, both PETA+0% HA and PETA+20% HA demonstrated greater stability than other experimental materials tested, with losses similar to those of the PCL control. The composites of the present invention have greater physiological stability than that of previously reported composites, which is an advantage since bones typically take weeks or months to regenerate. TMPETA-containing polymers and composites degraded much more rapidly than those with PETA, and their stability correlated with the molecular weight of the oligomer.

FIG. 3 shows that the PETA+20% HA foam and solid had significantly greater mass loss than the PCL control foam. Without wishing to be bound by this hypothesis, the mass loss is believed to result from hydrolytic chain scission in a manner similar to the degradation of PCL in physiological solutions. The PCL foam sample slightly increased in mass, perhaps from mineralization or non-specific protein deposition.

### Example 17

### Cytotoxicity Test Results

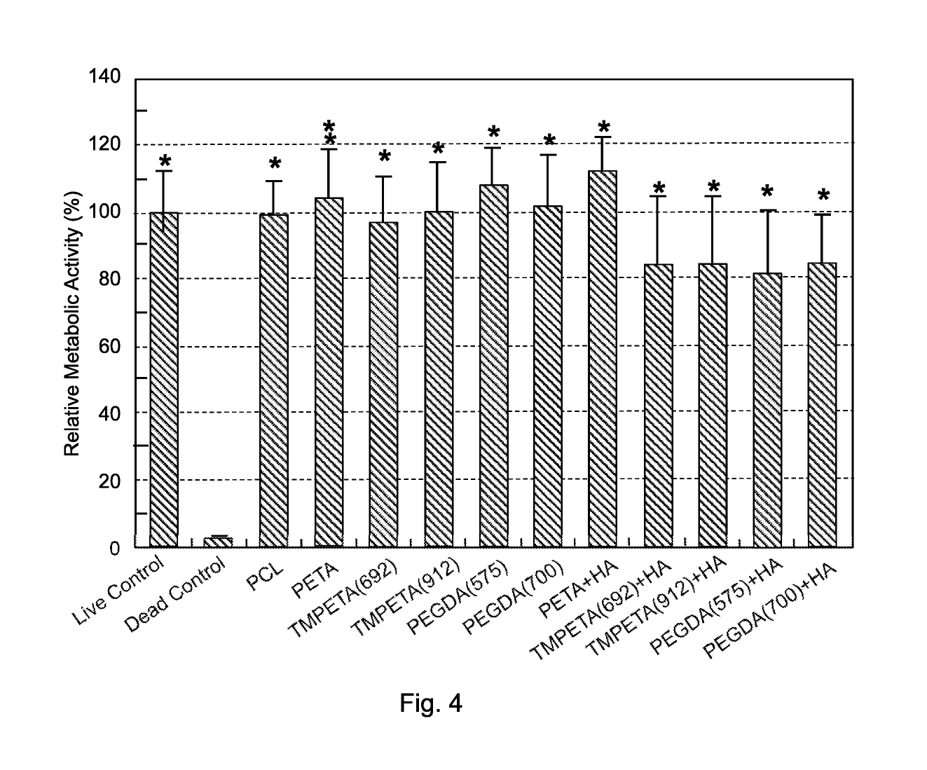

FIG. 4 shows the relative metabolic activity of hASC in thiol-acrylate extracts as measured by AlamarBlue fluorescent conversion. Relative fluorescent units are normalized versus live control. Asterisks indicate a sample that is significantly different from dead control. The conversion of AlamarBlue by PETA+0% HA and PETA+20% HA polymers was statistically not distinguishable from the tissue culture treated polymer and PCL control samples. It was also similar to the other materials tested.

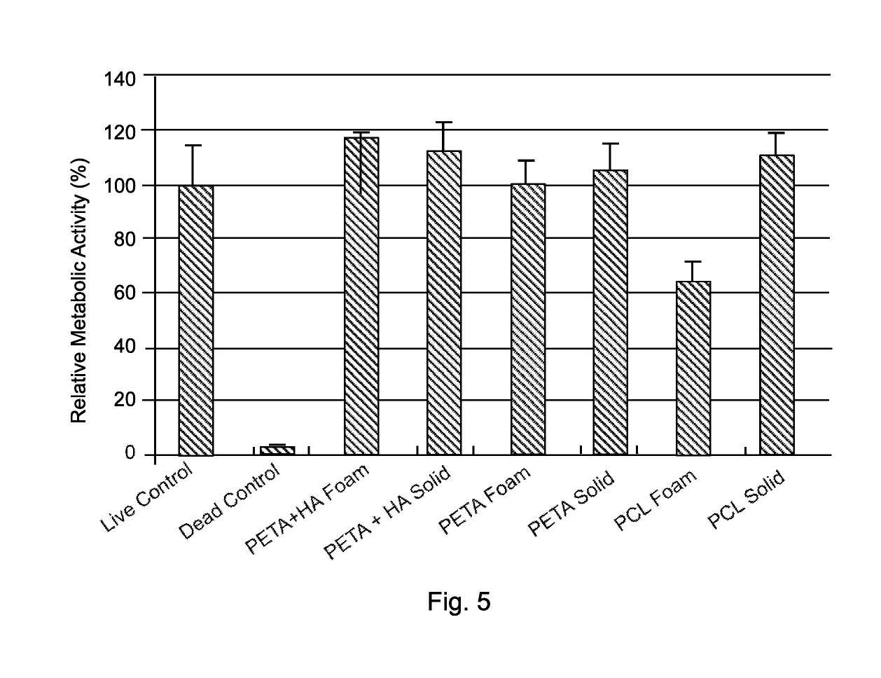

Tissue culture-treated polystyrene served as the positive control, while ethanol-treated hASC served as a negative control. Cells exposed to both solid and foam PETA+0% HA and PETA+20% HA composite extracts had significantly higher metabolic activity than the dead control or cells exposed to the PCL foam extract (FIG. 5). Based on the mass loss results, the significant reduction in metabolic activity of hASC cultured with PCL extracts did not correlate with an increased reduction in mass of PCL scaffolds. This observation suggests that the extraction products from the novel copolymers should be less toxic than extracts from PCL products.

### Example 18

### Mechanical Test Results

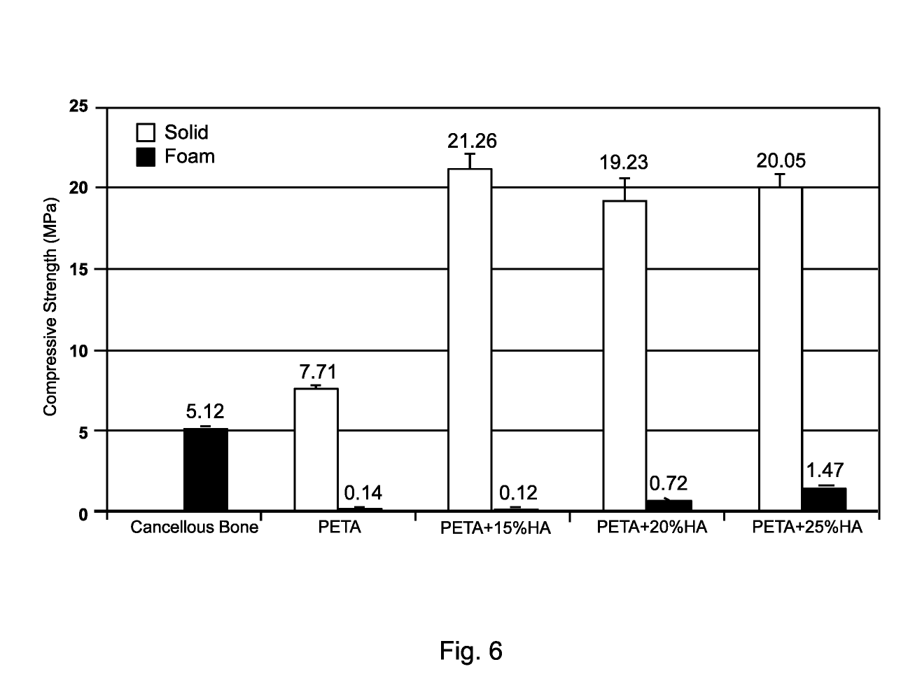

FIG. 6 shows measured compressive yield strengths in mechanical tests of foamed and solid PETA composites. Human cancellous bone was the control, measured at 5.12 MPa. The compressive strength of foamed PETA samples steadily increased with increasing HA content. However, the solid samples behaved differently. The addition of 15% HA resulted in a substantial increase in mechanical strength, but there was no significant change in yield strength for solid samples with higher concentrations of HA.

All else being equal, a foamed polymer will generally have lower mechanical strength than a solid polymer due to its porosity. The porosity is presumably responsible for the differing trends seen in solid and foamed scaffolds as HA content increases. Increasing HA content reduced pore size, and the decreased pore volume resulted in a more solid-like and stronger structure in the foam samples. As porosity plays little or no role in the solid (non-foamed) samples, the HA content had less of an effect their mechanical strength.

We also observed that the mechanical properties of the copolymer were very similar for in vitro polymerization (0.84±0.05 MPa), and for in situ polymerization in physiological media (0.85±0.06 MPa).

### Example 19

### Biocompatibility and Metabolic Activity Results

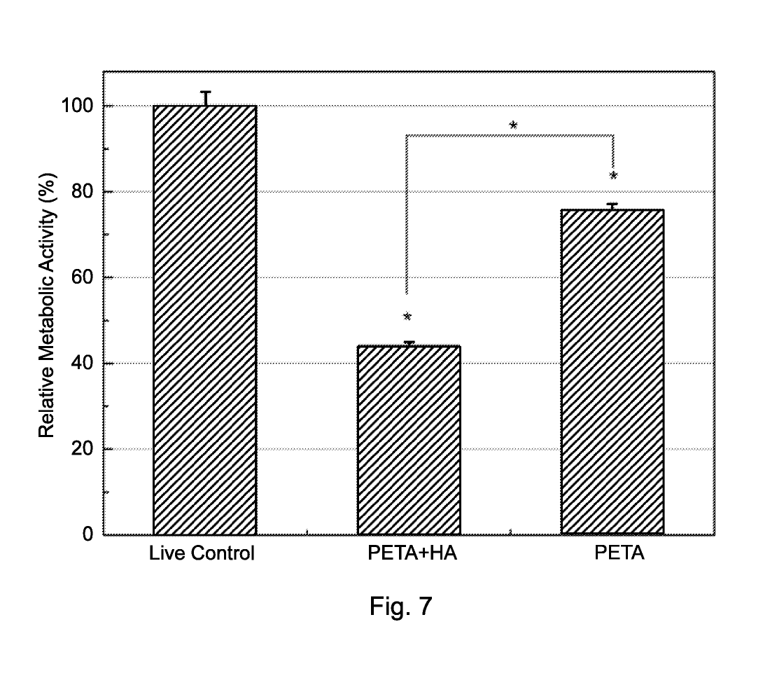

Relative metabolic activity for hASC cultured on solid substrates is shown in FIG. 7. The results are normalized versus the live control. Asterisks indicate samples that were significantly different from live control. The PETA samples and the PETA+HA samples were also significantly different from each other. Cells were cultured on solid PETA composite samples for 7 days in stromal media and assayed for fluorescent AlamarBlue conversion; polystyrene treated tissue culture plates served as a positive control. Compared to the positive control, hASC cultured on both PETA scaffolds had significantly lower metabolic activity. Further, cells cultured on the PETA+20% HA composite had significantly lower metabolic activity than cells cultured on the PETA+0% HA sample. Without wishing to be bound by this hypothesis, this observation may reflect reduced the metabolic activity that is associated with the differentiation of stem cells exposed to HA, a known osteogenic compound; it does not necessarily reflect reduced biocompatibility.

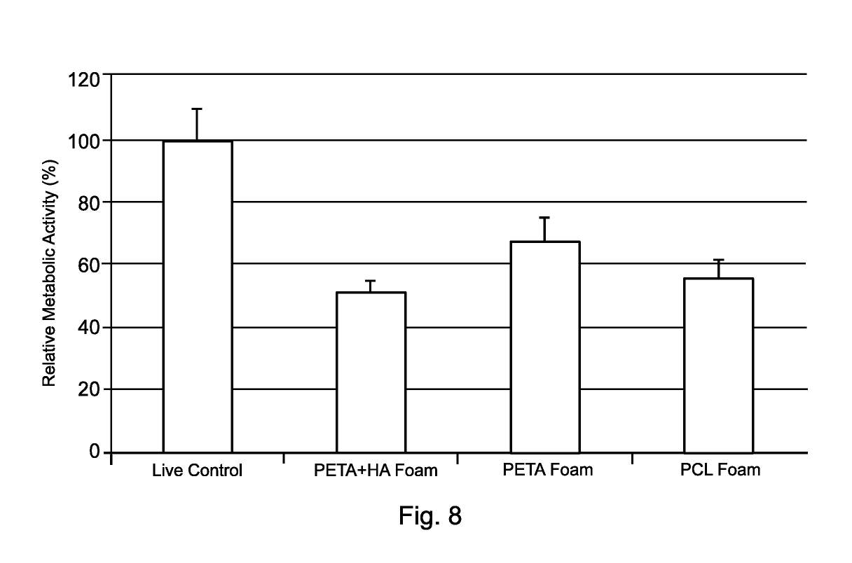

Relative metabolic activity for hASC cultured on foamed samples is shown in FIG. 8. The results are normalized versus the live control. PETA+0% HA foam demonstrated higher metabolic activity than both the solid PETA+20% HA composite and the PCL+0% HA foam, but significantly lower metabolic activity than cells on tissue culture treated styrene (the live control). Although the foamed PETA scaffold had a substantially larger surface area than that of the solid PETA scaffold, the results indicated that both forms of PETA supported hASC growth at levels similar to that of the PCL positive control.

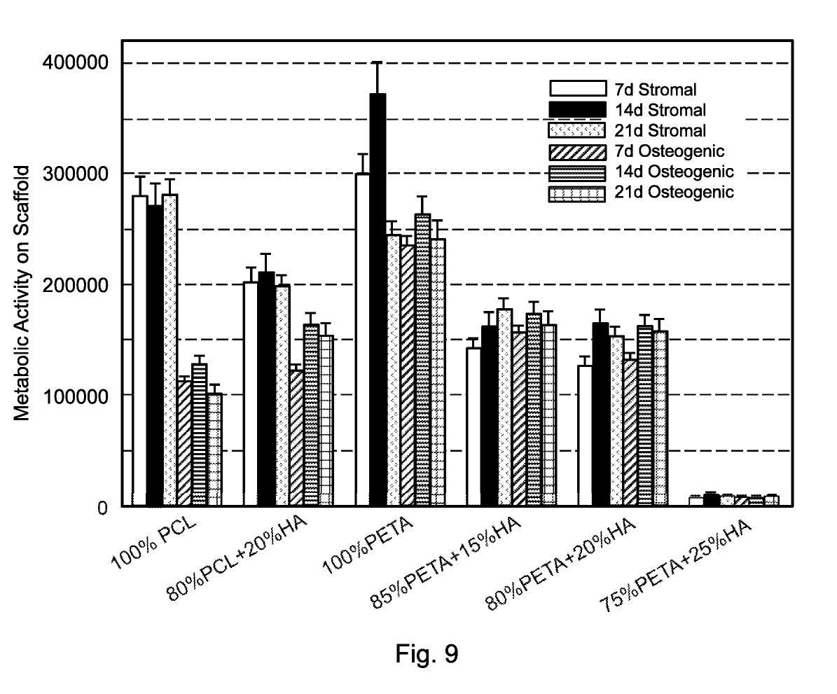

The hASC were cultured under osteogenic and stromal control media for 21 days. Relative levels of AlamarBlue conversion, indicating metabolic activity, are shown in FIG. 9 (depicting relative intensity units). Among all the scaffolds, PETA+0% HA showed highest metabolic activity, followed by PCL+0% HA. Almost no metabolic activity was measured in PETA+25% HA scaffolds, likely a result of smaller pore size and interconnectivity, reducing the number of viable cells within the scaffold. When HA was added either to PCL or PETA scaffolds, decreased metabolic activity was observed in stromal media but not in osteogenic media.

In FIG. 9, PCL control scaffolds with and without HA showed significant differences in metabolic activity at all time points. The PETA+0% HA control also showed significant differences at 7 and 14 days. PETA+15/20/25% HA samples showed no significant differences in metabolic activity between stromal and osteogenic conditions. The reduced metabolic activity is common in osteogenic differentiation of mesenchymal stromal cells. Cells in osteogenic media would likely be differentiating regardless of the ceramic content of the scaffold, and therefore little or no difference in metabolic activity would be expected.

### Example 20

### Calcium Deposition Results

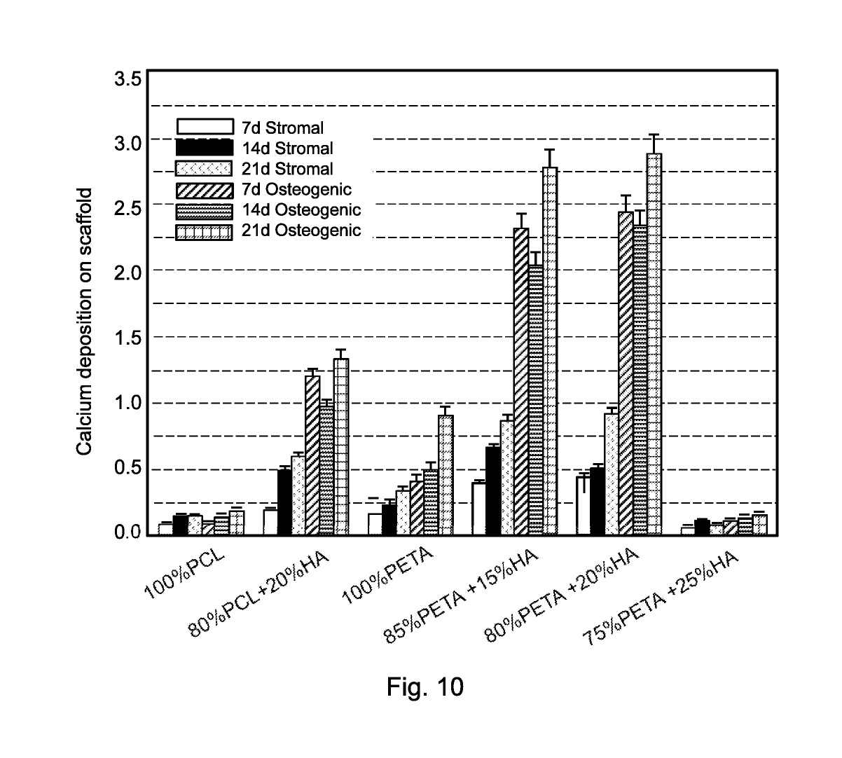

FIG. 10 illustrates calcium deposition on cells cultured on the PETA composites in stromal and osteogenic media, with PCL samples acting as controls. As expected, alizarin red staining tended to be considerably higher for hASC cultured in osteogenic media as compared to samples cultured in stromal media.

The hASC cultured in osteogenic media showed a significant increase in staining with increased time in culture. Significant differences in calcium deposition were observed at 14 days between stromal and osteogenic media among all the scaffolds, except for PETA+25% HA. Both PETA+15% HA and PETA+20% HA showed increased staining at 21 days, significantly more than the PETA control. However, almost no calcium deposition was observed at 14 or 21 days for PETA+25% HA.

Cross-section images of the scaffolds (not shown) confirmed that the PETA+25% HA sample had poor interconnectivity, with almost no alizarin red penetrating the interior of the scaffold. The PETA+25% HA scaffolds lacked sufficient nutrient and waste transport capability to support substantial cell growth.

### Example 21

### Quantification of DNA Results

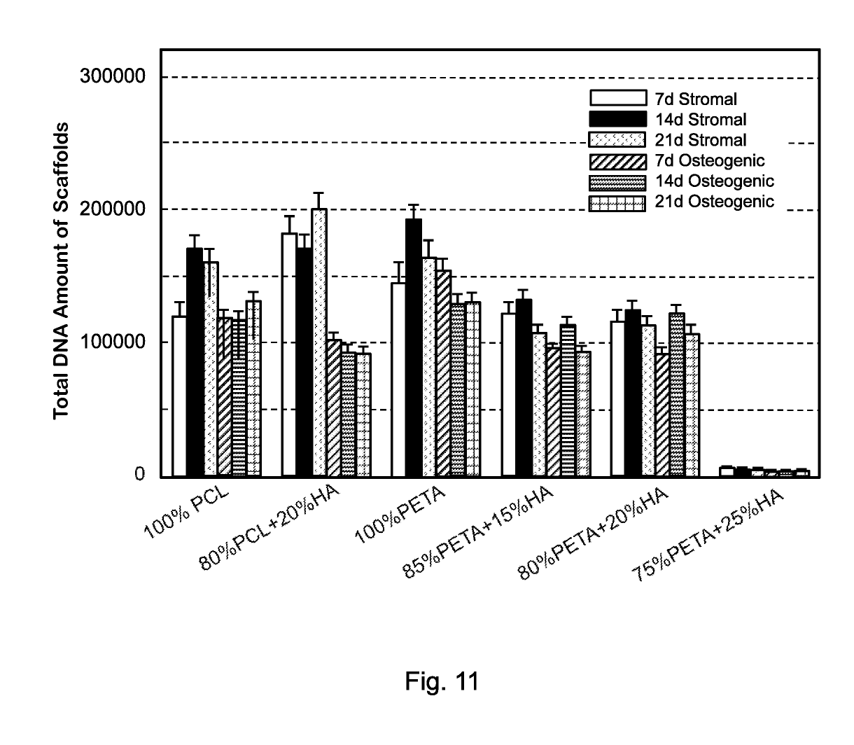

Total DNA content was determined using PicoGreen, to form a quantitative measurement of hASC proliferation within the scaffolds. Results are shown in FIG. 11. Differences in DNA content were observed between stromal and osteogenic media at 7 days for PCL+20% HA, PETA+15% HA, and PETA+20% HA composite scaffolds. The most pronounced difference occurred with the PCL+20% HA composite. At 14 and 21 days, significant differences in DNA content were observed for both PCL scaffolds as well as for the PETA+0% HA scaffold, but not for the PETA+15/20/25% HA composite scaffolds.

In comparing samples with the stromal media treatments, the PETA+15/20/25% HA samples had significantly fewer cells than did the PCL+0% HA or PETA+0% HA controls. Within the osteogenic media treatment groups, there were slight differences between the HA-containing and control scaffolds, but all scaffolds had a similar numbers of cells. These findings indicated decreased metabolic activity for the PETA+15/20/25% HA composite as compared to PETA+0% HA control. Without wishing to be bound by this hypothesis, this difference in cell function is likely attributable to osteogenic differentiation of hASC.

### Example 22

### Quantitative Reverse Transcriptase Polymerase Chain Reaction Results

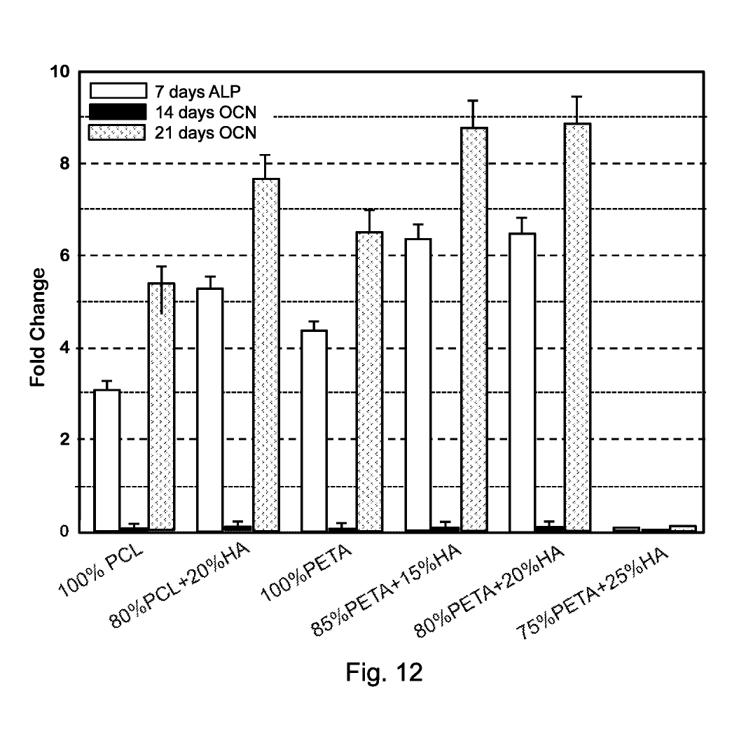

Several morphogenic proteins regulate osteogenesis. The morphogenic proteins act on transcription factors (such as core binding factor alphal), and cause the activation of osteoblast-related genes such as those for alkaline phosphatase (ALP) and osteocalcin (OCN). The expression levels of these genes were used as markers for early- and middle-stage osteogenesis, respectively. Quantitative reverse transcriptase polymerase chain reaction was used to assess the transcription of ALP mRNA at 7 days, and OCN at 14 and 21 days. The differences in the transcription levels of ALP and OCN on the varying scaffolds in stromal and osteogenic media are shown in FIG. 12.

At 7 days, the cells on PETA+15% HA and PETA+20% HA scaffolds showed similar levels of transcription of ALP, and both were significantly higher than transcription on other PETA and PCL scaffolds. Cells on PETA+0% HA scaffolds had higher ALP transcription than those on PCL+0% HA control scaffolds. While cells cultured on PCL+20% HA scaffolds had higher ALP transcription than those on pure PETA or PCL scaffolds, the transcription was still lower than for the PETA+15% HA and PETA+20% HA scaffolds.

Transcription of the OCN marker was low at 14 days, and increased substantially by 21 days with increasing hASC differentiation.

At 21 days, the OCN transcription in hASC as a function of scaffold type showed the same trend as ALP transcription, with cells cultured on PETA+15% HA and PETA+20% HA scaffolds demonstrating the greatest transcription of OCN. Cells cultured on the PETA+25% HA sample exhibited low transcription of both markers, likely the result of poor cell proliferation in the absence of an interconnected, porous structure within the scaffold.

### Example 23

### SEM Analysis

SEM images of PETA foam films (not shown) revealed a largely closed-cell structure, with a pore size ranging from ˜200-300 μm. Pores were rarely seen in SEM images (not shown) of the corresponding solid copolymer, although a few bubbles were present, perhaps from air introduced during the mixing procedure. The pore sizes in the foamed sample were similar to the pore sizes seen in native cancellous bone.

SEM images (not shown) of PETA solid polymer films after 7 days of hASC culture revealed that hASC adhered to the thiol-acrylate copolymer. The cells adhered well to the scaffold, and took on the typical spindle shape during culture. At lower magnification (100×) a confluent cell population was seen, spread more or less uniformly across the surface, while at higher magnification (1000×) the aligned spindle shape of individual cells could be clearly seen.

Without wishing to be bound by this hypothesis, it seems likely that the thiol groups impart a negative charge to the PETA copolymers, potentially increasing the adhesion, spreading, and proliferation of hASC on these surfaces compared to neutral surfaces.

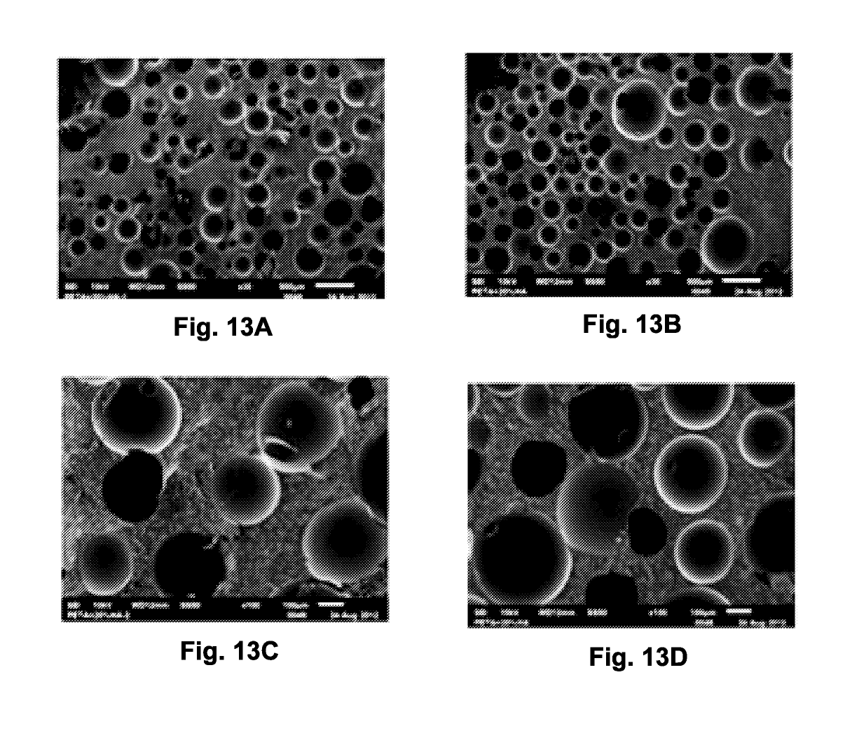

FIGS. 13A and 13C show SEM images of in vitro PETA+20% HA foam, and FIGS. 13B and 13D show SEM images of in situ PETA+20% HA foam. Scale bars are 100 μm in FIGS. 13A and 13B, and 10 μm in FIGS. 13C and 13D. SEM analysis indicated that there was no substantial difference in porosity or morphology between the in vitro and in situ foamed samples.

As seen in SEM images (not shown) of foamed scaffold samples, as HA concentrations increased, total pore volume and average pore size decreased. The morphology of the foam appeared most similar to cancellous bone around PETA+15% HA. An apparent transition was seen between 20% and 25% HA, with greatly reduced interconnected void volume. This change likely resulted from increased polymer solution viscosity, reducing the expansion of the nitrous oxide propellant and the mobility of the nitrous oxide bubbles. Similar morphological trends were seen in the micro-CT images.

### Example 24

### Micro-CT Analysis Results

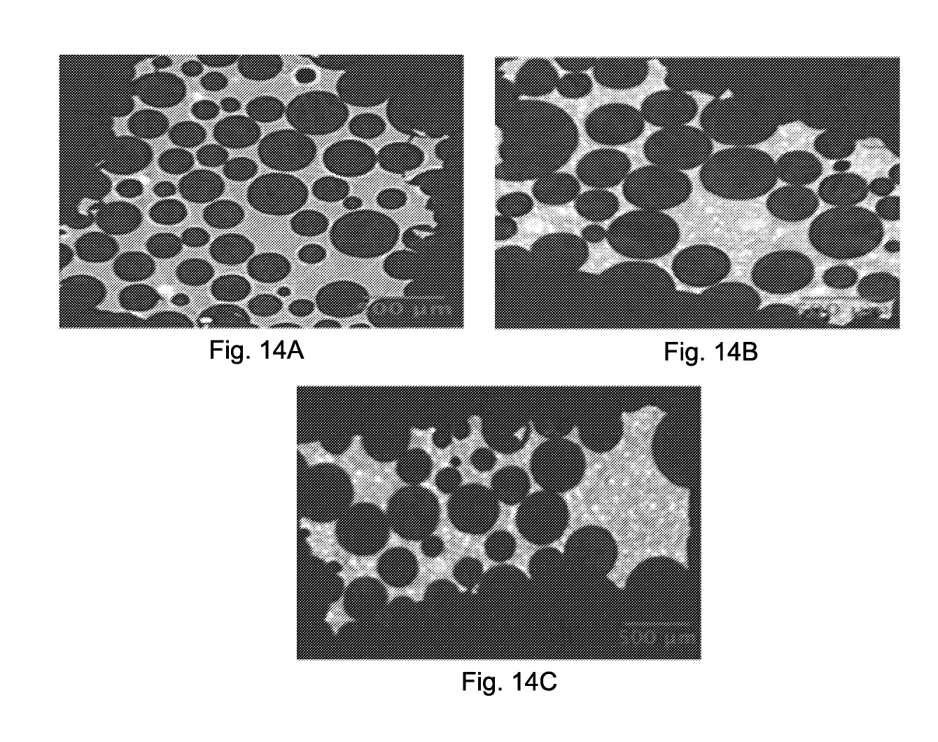

FIGS. 14A-C show micro-CT images of orthogonal slices of foamed scaffold samples analyzed using NIH ImageJ. The scale bars are 500 μm. The image data show good contrast between HA and polymer, confirming the suitability of micro-CT as an appropriate technique to image HA distribution and pore morphology. Measurements using ImageJ from these datasets indicated pores ranging from 100-500 μm for the PETA+0% HA control, and 125-800 μm for the PETA+20% HA. HA aggregations around 10-50 μm were seen; using a higher torque and a higher stirring speed may help improve homogeneity.

Three-dimensional micro-CT images (not shown) were used to visualize open cells and interconnectivity. These images showed that increasing the HA content resulted in smaller pore sizes and reduced porosity in the PETA composites. These effects were particularly evident at PETA+25% HA, for which the void spaces no longer appeared interconnected, and which had the appearance of a closed-cell foam. Increasing HA content above 25% did not appear to have much further effect on PCL void volume and interconnectivity. Because the PCL scaffold was synthesized by thermal precipitation, the pore size, volume, and interconnectivity are expected to be largely independent of solution viscosity. By contrast, increasing the HA content in PETA composite substantially affected viscosity, providing further support that the pore size and interconnectivity of the pore volume were affected by viscosity.

It is desirable to have an interconnected pore structure to support cell migration, cell differentiation, nutrient transport, and perhaps angiogenesis.

The step growth nature of the amine-catalyzed Michael addition reaction used in this invention essentially eliminates the chance that unreacted monomer or free radicals might leaching from the scaffold, as could occur if instead a free-radical, chain-growth polymerization were used. In situ polymerization permits absorbable foams to be used for conformal repairs of critical sized tissue defects, foams that can be easily delivered in a clinical or surgical setting.

Our SEM analysis and mechanical testing both showed that there was little difference between the PETA foams made in situ and in vitro. The novel foam represents a substantial improvement over prior PCL foams, which are formed externally prior to surgical insertion. The novel foams are also a substantial improvement over methyl methacrylate bone cements, which are largely inert, non-porous, and permanent.

### Example 25

### Certain Embodiments

In one embodiment the material is initially prepared as a two-component system, with the thiol and acrylate components in separate containers as part of an integrated delivery system. The acrylate-containing portion contains both the in situ tertiary amine catalyst, and the acrylate monomer without amine. When activated, the delivery system releases the thiol and acrylate components, mixes them in a static mixer, and injects the mixture under pressure into a tissue defect. The copolymer then forms a foam and cures in situ to fill the defect site.

In another embodiment the thiol and acrylate components are contained separately in the barrels of a two-part syringe, fitted with a static mixing apparatus. When the plunger is depressed, the thiol and acrylate materials are mixed and extruded into the tissue defect site where the copolymer can cure.

In another embodiment the thiol, acrylate, and amine in situ catalyst components are mixed in a container and poured or hand-filled into a defect site prior to curing.

### Example 26

### In Vivo Study

Scaffold Preparation and Surgical Implantation

Five male Fischer rats (Harlan Sprague-Dawley, Indianapolis, Ind.) were randomly assigned to three different treatment cohorts: (1) pre-sculpted (20% HA), (2) foamed in situ (20% HA), (3) foamed in situ (0% HA).

The autoclaved stock/HA and TMPTMP/HA pre-polymer mixture was placed in a 250 mL pressurized spray canister using 7 g compressed nitrous oxide as a gas foaming agent. The spray canister components were gas sterilized before surgical use. The foamed composite copolymer was expelled from the canister into a sterilized pan. The composite was cut into a rectangular prism with dimensions (15 mm×10 mm×1 mm) and placed into specimen (rat) 1.

For specimens 2, 3, and 4, the pre-polymer mixture was prepared as described above, but was foamed into a 5 mL syringe instead of the sterile pan. 3 mL of foam from the syringe was injected on either side of the spinal column of a rat with previous bilateral decortication of the L4 and L5 transverse processes. The same process was used for specimen 5, except that the foam contained 0% HA. The spinal column of specimen 3 was harvested at 3 weeks, while spinal columns from specimens 1, 2, 4, and 5 were harvested at 6 weeks.

To conduct posterolateral lumbar spinal fusion surgery on the rats, specimens were treated with a subcutaneous injection of 0.5 mg/kg butorphanol (Torbugesic, Fort Dodge Animal Health) and 0.02 mg/kg glycopyrrolate (Robinul-V, Fort Dodge Animal Health, Fort Dodge, Iowa). To induce anesthesia, specimens were administered isoflurane 20 minutes later in an induction chamber. The isoflurane was maintained at 1.5% via nose cone on a Bain circuit for the remainder of the procedure. The lumbar region was clipped and aseptically prepared with 70% isopropanol/betadine. A posterior midline skin incision was made over the lumbar spine. Two fascial incisions were made 3 mm lateral and parallel to the spinous processes. The L4 and L5 transverse processes were exposed using a combination of sharp and blunt dissection that was limited to the specific area of interest. A scalpel was used to decorticate the transverse processes bilaterally. The surgical sites were thoroughly lavaged with physiologic saline. Based on cohort assignment, scaffolds were placed on both sides of the spine such that they spanned between the midpoint of each transverse process. Fascial and subcutaneous incisions were closed separately with 3-0 polyglactin 910 (Vicryl, Ethicon) in a simple continuous pattern. To inhibit migration, closure of the fascia around the implants effectively filled any potential space. Subcutaneous tissue was apposed similarly. Tissue adhesive was used for skin closure. (Vetbond, 3M). The specimens were humanely euthanized by CO₂ asphyxiation 3 or 6 weeks after surgery.

Micro-CT Analysis

Spines were harvested immediately postmortem. Two-dimensional (2-D) micro computed tomography (μ-CT) imaging was performed with 0.04 mm slice widths. The settings were 40 kV, 540 ms, 360° rotation, with imaging at 0.9° steps (SkyScan 1074, Skyscan n.v., Belgium). Three-dimensional (3-D) files were reconstructed based on a compilation of 2-D images. Measurements of 2-D and 3-D images were performed with AVIZO software packages.

The density of the defect site was tested using an x-ray line scanning method with Image J. A ventral view of each spine was uploaded into Image J and analyzed with a density measuring tool. A density line was drawn on the contralateral tissue region unaffected by the surgery or treatment, to act as a positive control. The same density line was used to calculate the optical density (OD) of an area in the blank space of the image to serve as a negative control. The density line was then moved to the defect site. Using the same density line allowed a comparison of the optical density of the defect site with the positive and negative controls. A ratio of each mean OD was used to normalize the percentage of bone growth in each specimen. Each recorded density value represented the difference between the measured OD value and the OD for the blank space.

Histology

Specimen 3 (PETA:HA, 80:20, foamed in situ) was sacrificed three weeks post-operative. The spinal column containing the defect was removed. The spinal column was decalcified, and medial slices were cut and stained with Hematoxylin and eosin.

Specimen 1 (pre-sculpted PETA:HA, 80:20), Specimen 2 (PETA:HA, 80:20, in situ), Specimen 4 (PETA:HA, 80:20, in situ), and Specimen 5 (PETA:HA, 100:0, in situ) were each sacrificed at 6 weeks post-operative. The spinal column from each specimen was removed, decalcified, and stained as otherwise described for specimen 3.

In Vivo Study

### 3.7.1 Radiography

All rat specimens survived surgery plus up to 6 weeks post-op. Observed behavior and weight gain (90.7±5.9 g) were normal for all rats. Rats that were injected with the foamed thiol-acrylate nanocomposite showed some calcification at 3 weeks after implantation, which became more evident at 6 weeks after implantation (44.1% bone formation). The increasing intensity of bone scaffolds indicated ossification of tissue intercalated into the scaffolds. (X-ray radiographs are not shown here, because they typically reproduce poorly in published patent documents.) Rats implanted with pre-molded samples, however, had a much lower increase in intensity at 6 weeks after implantation (37.1% bone formation). No evidence of calcification was observed in the rat implanted with 0% HA pre-sculpted PETA scaffolds (8.1% bone formation). This last observation was consistent with our earlier, in vitro osteogenesis study, which found that the scaffolds without HA did not significantly induce expression of osteogenic markers as compared to control.

Micro Computed Tomography

Micro-CT was also performed to analyze bone formation. Avizo software was used to render micro-CT data. (The CT images are not shown here, because they typically reproduce poorly in published patent documents.) The CT images were generally consistent with the radiography results. Further, the rats injected with foamed in vivo scaffolds showed regions of discontinuous ossification in the scaffold area 6 weeks post-surgery. During the surgery, the structure of foamed in vivo sample may have been disrupted when the surgical site was closed. Reduced porosity and interconnectivity could then be the reason why new bone formation was seen to be non-continuous. Thus it is preferred that the surgical procedures used not be allowed to disrupt the foam; or that the foam be adequately cured before the surgical site is closed; or both.

### 3.7.3 Analysis of Bone Formation Using Histology

Histology was analyzed for on the spinal defect from rat 3 (PETA:HA 80:20, foamed in situ) after sacrifice at 3 weeks. Decalcifying and staining the spinal column posed a practical problem in analyzing tissues for bone formation. For this reason, the structure of the tissues and the presence of osteoblasts were used to assess bone formation via histology. These images (also not shown here, because they typically reproduce poorly in published patent documents) showed early signs of cell organization and osteoblast formation. Rat 3 provided a comparison for assessing the other treatments. Lymphocytes were observed proximal to the implant at three weeks post-operative, but not in the rats six weeks post-operative.

Rat 1 (pre-sculpted PETA:HA 80:20) was analyzed at six weeks. Significant signs of organized tissue were seen in comparison to Rat 3 at three weeks. The presence of osteoblasts in the tissue suggested bone growth at the defect site. (Bone per se was not seen due to the decalcification step employed.) Early signs of blood vessel formation were also seen, indicating that the implants were compatible with the surrounding tissue, and that they promoted tissue in-growth.

Images from the implants in rats 2 and 4 (PETA:HA 80:20 in situ) showed large areas of organized tissues containing osteoblasts. Well-defined osteoblasts indicated bone formation, and confirmed the regenerative properties of the novel implants. There was little indication of lymphocytes in the tissues, meaning that the scaffolds induced little inflammatory response. The ingrowth of tissue into the PETA:HA (80:20) scaffold that was foamed in situ was not significantly different from that seen for the pre-sculpted (PETA:HA 80:20) composite. This observation supports the conclusion that the in situ polymerization process did not induce significant cytotoxicity in the surrounding tissue, nor adversely affect the biocompatibility of the composite material.

Histological analysis of images from rat 5 (PETA:HA 100:0) indicated that the absence of nanoscale HA particles reduced the osteogenic properties of the scaffolds. There was a substantial reduction in the formation of organized tissue and cellular attachment to the implants. However, this polymer-only sample still possessed general biocompatibility; some areas of the implant were closely associated with organized tissues, while in other areas the tissue appeared to be separated from the underlying polymer.

The complete disclosures of all references cited throughout the specification are hereby incorporated by reference in their entirety, as is the complete disclosure of priority application Ser. No. 61/721,607. Also incorporated by reference is the complete disclosure of Bounds, C. O.; Upadhyay, J.; Totaro, N.; Thakuri, S.; Garber, L.; Vincent, M.; Huang, Z.; Pojman, J. A. “Fabrication and Characterization of Stable Hydrophilic Microfluidic Devices Prepared Via the in Situ Tertiary-Amine Catalyzed Michael Addition of Multifunctional Thiols to Multifunctional Acrylates,” ACS Appl. Mater. Interfaces, 2013, 5, 1643-1655. In the event of an otherwise irresolvable conflict, however, the disclosure of the present specification shall control.

What is claimed:

1. A method for synthesizing a copolymer, said method comprising the steps of:
(a) Reacting a monomer selected from the group consisting of an alkyne comprising at least two alkyne groups, and an acrylate comprising at least two acrylate groups; with a nitrogenous compound selected from the group consisting of ammonia, a primary amine, a secondary amine, and a tertiary amine; and continuing the reaction until between about 0.1% and about 10% of the monomers are converted into tertiary amines; whereby a mixture results that contains both unreacted monomer and tertiary amine;
(b) reacting the mixture resulting from step (a) with a thiol comprising at least two thiol groups; wherein the tertiary amine acts as a catalyst to catalyze the copolymerization of the thiol with the monomer to form a thiol-acrylate copolymer or a thiol-alkyne copolymer.

2. The method of claim 1, wherein the monomer comprises an acrylate comprising at least two acrylate groups.

3. The method of claim 1, wherein the monomer comprises an alkyne comprising at least two alkyne groups.

4. The method of claim 1, wherein one or more of the following conditions is true: at least some of the nitrogenous compound comprises ammonia, or at least some of the monomer comprises at least three acrylate groups, or at least some of the thiol comprises at least three thiol groups; and wherein some degree of crosslinking occurs during the copolymerization step.

5. The method of claim 1, wherein the nitrogenous compound comprises a secondary amine, the monomer comprises three acrylate groups, and the thiol comprises three thiol groups; and wherein some degree of crosslinking occurs during the copolymerization step.

6. The method of claim 5, additionally comprising the step of entraining gas bubbles into the mixture during the copolymerization step, so that the resulting copolymer is porous.

7. The method of claim 6, additionally comprising the step of incorporating hydroxyapatite crystals into the mixture before or during the copolymerization step, so that a composite material is produced in which the porous copolymer contains multiple inclusions of hydroxyapatite crystals.

8. The method of claim 7, wherein the copolymerization step occurs in situ and in vivo within damaged or diseased bone tissue.

9. The method of claim 8, additionally comprising the step of allowing new bone tissue to grow in vivo, using the composite material as a scaffold for growth.

10. The copolymer produced by the method of claim 1.

11. The copolymer produced by the method of claim 2.

12. The copolymer produced by the method of claim 3.

13. The copolymer produced by the method of claim 4.

14. The copolymer produced by the method of claim 5.

15. The copolymer produced by the method of claim 6.

16. The composite material produced by the method of claim 7.

17. A thiol-acrylate copolymer, wherein the ratio of thiol to acrylate is 1:1.

18. A thiol-alkyne copolymer, wherein the ratio of thiol to alkyne is 1:1.
